(19) United States
(12) Patent Application Publication
(10) Pub. No.: US 2023/0293754 A1
(43) Pub. Date: Sep. 21, 2023

(54) Pathogen Transfer Prevention and Mitigation Apparatuses

(71) Applicant: Lucian Randolph, Oviedo, FL (US)

(72) Inventor: Lucian Randolph, Oviedo, FL (US)

(21) Appl. No.: 18/024,071

(22) PCT Filed: Sep. 1, 2021
(86) PCT No.: PCT/US2021/048735

§ 371 Date: Mar. 1, 2023

Related U.S. Application Data
(60) Provisional application No. 63/073,173, filed on Sep. 1, 2020.

(51) Int. Cl. A61L 2/24; A61L 2/14; A61L 2/22; A61L 2/20; A61L 2/025; A61L 2/10; B03C 3/38; B03C 3/41; B03C 3/68; B03C 3/47

(52) U.S. Cl. CPC A61L 2/24; A61L 2/14; A61L 2/22; A61L 2/20; A61L 2/025; A61L 2/10; B03C 3/38; B03C 3/41; B03C 3/68; B03C 3/47; A61L 2202/14; A61L 2202/11; A61L 2202/15; A61L 2202/13

(57) ABSTRACT

Disclosed herein are embodiments of an invention relating to pathogen transfer mitigation and prevention apparatuses. Described herein are embodiments comprising one or more charged particle emitters, collectors, power circuits, and controllers. The invention described herein can effectively, for example, prevent, stop, and/or minimize the transfer of pathogens such as, for example, viruses, bacteria, fungi, protozoa, and/or worms. The invention described herein has application in many fields, including, for example, the agriculture, restaurant, food, livestock, pet, sports, entertainment, travel, and/or transportation industries. The invention described herein is of particular relevance given the recent and ongoing international coronavirus disease (COVID) pandemic.

This application claims priority to U.S. Provisional Application No. 63/073,173 filed Sep. 1, 2020, which is incorporated in its entirety by reference.

### BACKGROUND

The present invention relates to apparatuses for the prevention and mitigation of pathogen transfer, especially regarding pathogens transmitted through the air. Such apparatuses are useful in any environment where it is desirable, for example, to prevent, block, stop, or minimize transmission of potentially harmful or infectious pathogens. Such pathogens can include, for example, viruses, bacteria, fungi, protozoa, or worms. Such environments can include, for example, industries or businesses that grow living organisms, such as, plants or animals. Examples include the agriculture, restaurant, food, livestock, or pet industries. Such environments can also include, for example, industries or businesses where humans congregate. Examples include conventions, conferences, or sports, entertainment, travel, voting, or transportation centers or hubs. Such environments can further include the air purification, filtration, and/or cleaning industries and/or markets. The present invention is particularly useful in light of the recent and ongoing international coronavirus disease (COVID) pandemic and the widespread and significant harms caused by the severe acute respiratory syndrome coronavirus 2 (SARS-CoV-2).

The present invention provides for the use of one or more charged particle emitters and collectors to stop and/or curb the transfer of surrounding or nearby pathogens. Charged particles bind and/or neutralize pathogens. Emitting charged particles to bind and/or neutralize pathogens, and/or collecting charged particles that have bound and/or neutralized pathogens, effectively prevents and mitigates pathogen transfer.

Current devices that utilize charged particle emitters and/or collectors cannot adequately mitigate or prevent pathogen transfer. Current devices are either closed devices that have a constant output or stream of charged particles and a closely spaced collector or open devices that provide an emitter only and not a collector. Closed devices are not suitable for use in wide spaces. Open devices are also ineffective due to interference effects that are inherent to charged particle physics, and familiar to those of ordinary skill in the art, and because they rely on comparatively slow gravitational effects as opposed to particle acceleration from the emitters, collectors, and/or surrounding particles. There is a need for an apparatus that can use charged particles across a wide space and that overcomes interference effects.

The present invention can mitigate and prevent pathogen transfer and be used across wider spaces. The enhanced range and efficacy allows for effective pathogen transfer mitigation and prevention in many settings as described herein.

Other features of the present invention will be apparent to those of ordinary skill in the art in light of this disclosure.

That certain U.S. provisional patent application No. 63/073,173 filed and received 2020 Sep. 1 is hereby incorporated by reference in its entirety for all that it contains (including all references incorporated by reference therein) for all purposes as if fully set forth herein to the maximum extent allowable by law.

Any and all publications, patents, patent applications, and references cited or referred to herein are hereby incorporated by reference in their entirety for all that they contain (including all references incorporated by reference therein) for all purposes as if fully set forth herein to the maximum extent allowable by law as if each individual publication, patent, patent application, or reference cited or referred to herein was specifically and individually indicated to be hereby incorporated by reference in its entirety for all that it contains (including all references incorporated by reference therein) for all purposes as if fully set forth herein to the maximum extent allowable by law.

### SUMMARY

The disclosed apparatus can comprise at least one emitter of charged particles, at least one controller capable of activating the one or more emitters in a temporal and/or spatial pattern, and at least one collector to collect particles emitted by the one or more emitters.

### BRIEF DESCRIPTION OF THE DRAWINGS

For purpose of explanation, several embodiments are set forth in the following figures, wherein:

FIG. 1 is a schematic block diagram of an embodiment of a pathogen transfer prevention and mitigation apparatus according to the present disclosure in a vertical multi-unit configuration;

FIG. 2 is a schematic block diagram of an embodiment of a pathogen transfer prevention and mitigation apparatus according to the present disclosure in an oblique multi-unit configuration;

FIG. 3 is a schematized example temporal and spatial pattern of pulsed emission according to the present invention;

FIG. 4 is a schematized example temporal and spatial emitter pattern of quadrilaterals changing in size according to the present invention;

FIG. 5 is a schematized example temporal and spatial emitter pattern of lines moving according to the present invention;

FIG. 6 is a schematized example temporal and spatial emitter pattern of lines rotating according to the present invention;

FIG. 7 is a schematic block diagram of an embodiment of a pathogen transfer prevention and mitigation apparatus according to the present disclosure in a freestanding assembly; and

FIGS. 8A-D are schematic block diagrams of components and processes of an embodiment of a pathogen transfer prevention and mitigation apparatus according to the present disclosure.

### DETAILED DESCRIPTION OF PREFERRED EMBODIMENTS

In the following description, details are set forth for purpose of explanation. However, one of ordinary skill in the art will realize that the embodiments described herein may be practiced without the use of all of these specific details. The embodiments (and descriptions) disclosed herein are intended, therefore, to be illustrative only and not limiting. Similarly, where examples are used herein, the examples are not intended to be limiting unless the context in which the example is used clearly indicates otherwise. Accordingly, “for example” or “e.g.” should be read as “for example, and without limitation,” unless the context indicates that limitation to the given example(s) is intended.

Herein, “operable” or “adapted” means configured, sized, positioned, and/or arranged as appropriate to render an item suitable for use with another apparatus. “Operable” or “adapted” is intended herein as a description of structure and not as a description of function.

The meaning of other terms may be defined herein or will otherwise be apparent to those of ordinary skill as the ordinary meanings used in the art.

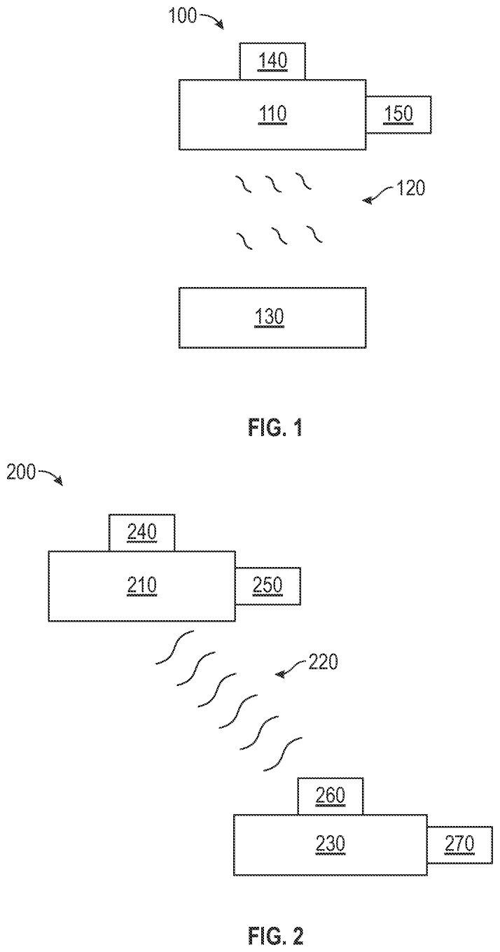

Referring to FIG. 1, an embodiment of an apparatus for the prevention and mitigation of the transfer of pathogens is shown. Apparatus 100 comprises at least one charged particle emitter 110, at least one charged particle collector 130, at least one emitter power circuit 140, and at least one emitter controller 150. Emitter 110 emits charged particles 120, collector 130 collects charged particles including charged particles 120, emitter power circuit 140 powers emitter 110, and emitter controller 150 is operable to activate and deactivate emitter 110 in one or more emitter patterns that are temporal, spatial, or a combination thereof. Charged particles discussed herein, such as charged particles 120, may comprise oxygen radicals, hydroxyl ions, superoxide ions, dry fog, ultra-dry fog, negative air ions, nanometer sized negative air ions, charged water particles, nanoclusters of charged water particles, or a combination thereof.

Further embodiments may comprise multiple emitter elements, multiple collector elements, multiple emitter power circuit elements, and/or multiple emitter controller elements. Each of these elements can be variably or similarly sized relative to one, some, or all of the others. Each of these elements may be connected and/or related to one, some, or all of the others, in varying combinations, whether physically, electrically, magnetically, mechanically, temporally, spatially, or otherwise, or a combination thereof. For example, in apparatus 100, emitter 110 is physically connected to emitter power circuit 140 and emitter controller 150 but physically disconnected from collector 130.

FIG. 2 illustrates an additional pathogen transfer prevention and mitigation apparatus embodiment. Apparatus 200 comprises at least one charged particle emitter 210, at least one charged particle collector 230, at least one emitter power circuit 240, at least one emitter controller 250, at least one collector power circuit 260, and at least one collector controller 270. Emitter 210 emits charged particles 220, collector 230 collects charged particles including charged particles 220, emitter power circuit 240 powers emitter 210, collector power circuit 260 powers collector 230, emitter controller 250 is operable to activate and deactivate emitter 210 in one or more emitter patterns, and collector controller 270 is operable to activate and deactivate collector 230 in one or more collector patterns. The one or more emitter patterns and/or the one or more collector patterns may be temporal, spatial, or a combination thereof.

Further embodiments may comprise multiple emitter elements, multiple collector elements, multiple emitter power circuit elements, multiple collector power circuit elements, multiple emitter controller elements, and/or multiple collector controller elements. Each of these elements can be variably or similarly sized relative to one, some, or all of the others, in varying combinations. Each of these elements may be connected and/or related to one, some, or all of the others, in varying combinations, whether physically, electrically, magnetically, mechanically, temporally, spatially, or otherwise, or a combination thereof. For example, in apparatus 200, emitter 210 is physically connected to emitter power circuit 240 and emitter controller 250 but physically disconnected from collector 230, collector power circuit 260, and collector controller 270, while collector 230 is physically connected to collector power circuit 260 and collector controller 270 but physically disconnected from emitter 210, emitter power circuit 240, and emitter controller 250.

Additionally, in apparatus 200, emitter power circuit 240 is electromagnetically isolated from controller power circuit 260. In preferred embodiments, emitter power circuit elements are electromagnetically isolated from collector power circuit elements. Power isolation may improve safety when higher charges are used to attract particles across larger distances.

In certain embodiments, the one or more emitters comprise one or more oxygen radical emitters, hydroxyl ion emitters, superoxide ion emitters, dry fog emitters, ultra-dry fog emitters, emitters of negative air ions, emitters of nanometer sized negative air ions, emitters of charged water particles, emitters of nanoclusters of charged water particles, ultrasonic cavitation means, physical vaporization means, micro water steam impacting means, plasma evaporation means, pulsed laser evaporation means, ionization means, tuned ultraviolet light, carbon fiber ion generators, or a combination thereof. In certain embodiments, the one or more collectors comprise one or more oxygen radical collectors, hydroxyl ion collectors, superoxide ion collectors, dry fog collectors, ultra-dry fog collectors, collectors of negative air ions, collectors of nanometer sized negative air ions, collectors of charged water particles, collectors of nanoclusters of charged water particles, or a combination thereof.

In some embodiments, the one or more collectors comprise one or more collector plates that are removable, swappable, cleanable, or a combination thereof. In certain embodiments, the one or more collectors comprise one or more metallic sheets. In preferred embodiments, the one or more collectors comprise copper. In alternative embodiments, the one or more collectors comprise copper, aluminum, steel, iron, brass, bronze, zinc, nickel, graphene, copper alloy, aluminum alloy, bronze alloy, nickel alloy, iron alloy, wrought iron, cast iron, alloy steel, carbon steel, stainless steel, or a combination thereof. In certain embodiments, the one or more metallic sheets may be a strip. In certain embodiments, the one or more metallic sheets may be affixed to a floor, and it may be under a mat, carpet, tape, or a combination thereof.

Passive air ions and active precipitator particles have different physics and behavior with a primary difference being speed of the latter. Embodiments of the present invention generate unipolar particles and then bonds them to energy-cleaved water molecules, forming clusters 1-10 nanometers wide. Fields and gravity drive captured particulate matter onto collectors at up to 2 feet per second or faster, fast enough to eliminate virus aerosols from the active breathing zone immediately after being exhaled. Once on collectors, previously airborne contaminants are trapped and neutralized. When ions are generated from a coronal discharge emitter or a carbon fiber emitter, they are accelerated away from the emission point because of the electrostatic repulsion from the ions being generated behind them. In air, ions will travel about a foot accelerating and then they begin to dissipate and slow down. Over the next several feet, the ions slow to a crawl and begin to electromigrate. It is therefore desirable to create a downdraft of particles that cover a longer distance. The constant downdraft of charged particles accelerating to a collector can attack and remove aerosols or particulates driving them below breathing level. Creating a constant downdraft of charged particles from the emitters to the collectors that will quickly push exhaled aerosols from breath below the active breathing zone can thus protect others who are nearby breathing in the same airspace. By using the multipoint generated, uniformly-dispersed, electrostatic field of vertical-falling ion layers as an underlying principle, apparatuses for creating an overhead electrostatic field can be built in multiple configurations.

Embodiments of pathogen transfer prevention and mitigation apparatuses may comprise different quantitative combinations of the one or more emitters and the one or more collectors. Examples may include one emitter with one collector, multiple emitters with one collector, one emitter with multiple collectors, multiple emitters with a corresponding equal number of multiple collectors, or multiple emitters with a different number of multiple collectors. Embodiments may also comprise varying spatial arrangement and/or organization of the one or more emitters, the one or more collectors, and/or the one or more emitters in relation to the one or more collectors. The one or more emitters can be physically disconnected from the one or more collectors by purely vertical distance, such that the one or more emitters are “centered” above, the one or more collectors. For example, in apparatus 100, emitter 110 is centered above emitter 130. In certain embodiments, the one or more emitters can be positioned below the one or more collectors (not illustrated). The one or more emitters can be physically disconnected from the one or more collectors by both vertical and horizontal distance, such as the spatial relation of emitter 210 to collector 230 in apparatus 200. The one or more emitters can be physically disconnected from the one or more collectors by purely horizontal distance (not illustrated). In preferred embodiments, the one or more emitters are positioned above the one or more collectors, such that gravitational force can contribute to accelerating the rate of travel of the charged particles emitted by the one or more emitters and the charged particles collected by the one or more collectors.

Quantitative combinations of elements and spacing and/or positioning between elements may be permanent, temporary, stationary, moving, changing, or a combination thereof. Such combinations and positioning may also be uniform across elements or different between elements, and may be evenly or unevenly distributed among elements. Such combinations and positioning may contribute to the one or more emitter patterns and/or the one or more collector patterns of pathogen transfer prevention and mitigation apparatus embodiments.

In certain embodiments, the elements may be variably physically connected or disconnected to items in the environment. For example, one or more emitters and/or one or more collectors may be permanently or temporarily, directly or indirectly, affixed to one or more ceilings, walls, floors, or a combination thereof. In certain preferred embodiments, the one or more emitters are affixed to a mounting structure suspended from a ceiling or attached to a wall, and the one or more collectors are placed on or attached to a floor.

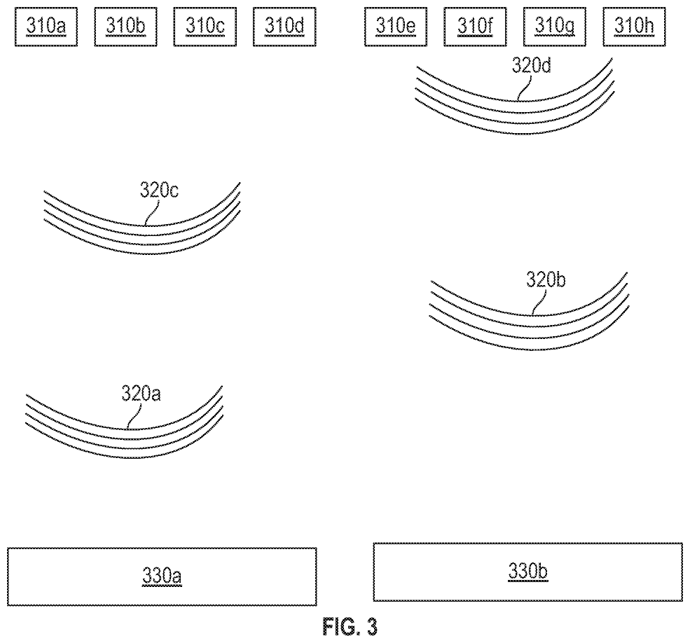

Turning now to FIG. 3, an example temporospatial emitter pattern is shown. In FIG. 3, charged particle emitters 310a, 310b, 310c, 310d, 310e, 310f, 310g, and 310h are arranged in a horizontal line physically disconnected by vertical distance from charged particle collectors 330a and 330b. Emitters 310a, 310b, 310c, and 310d are depicted emitting a first group of charged particles 320a, then emitters 310e, 310f, 310g, and 310h emit a second group of charged particles 320b, then emitters 310a, 310b, 310c, and 310d emit a third group of charged particles 320c, and then emitters 310e, 310f, 310g, and 310h emit a fourth group of charged particles 320d. Such a pulsatile pattern of emission allows for electric interaction, magnetic interaction, physical interaction, or a combination thereof between each group of charged particles and its surroundings, each of the other groups of charged particles, or a combination thereof. Such interactions can accelerate the rate of travel of the charged particles. Attraction and/or repulsion forces between charged particles that would slow the travel of charged particles across space can be overcome and/or harnessed by selective clustering of charged particles into smaller groups. In FIG. 3, group 320a, group 320c, and collector 330b are of the same positive or negative charge while group 320b, group 320d, and collector 330a are of the opposite charge. Thus, collector 330b and group 320a attract group 320b while group 320d repels group 320b, each of these interactions contributing to faster migration of group 320b to collector 330b. Groups of charged particles travelling in adjacent streams do not necessarily need to have opposite charge. In preferred embodiments, successive groups of charged particles within the same stream of motion are of the same charge, such that each new group of charged particles emitted repels and propels the one or more older groups of charged particles that were previously emitted. In preferred embodiments, the one or more groups of charged particles are of the opposite charge from the one or more collectors that would collect those charged particles, such that the one or more collectors attract those charged particles.

Forces acting on charged particles in an active particle field can include Brownian kinetic vibrational energy, electron transfer and accumulation, electrostatic field potential energy, electromagnetic field potential energy, electromagnetic force generated from relative movement of falling extra electrons in the charged particles, and/or electromotive force generated from falling magnetic moments in the charged particles. Individual ionized energy charges on each particle are the major contributor to the disruptive discharge effects. The size of the particle is also important. If a particle has enough mass, gravitational force pulling it downward can overcome Brownian motion effect that would tend to keep the particle afloat. Ions can be accelerated up to 2 meters per second by alternating repulsive and attractive forces. When combined with background gravitational pull and aimed downward, charged particles can be actively pushed and pulled downward, creating a relatively quick downdraft of charged air particles that is resistant to the drag effects of air, Brownian motion, or even moderate circulation.

Emitter patterns may comprise continuous emission, pulsatile emission, a pseudorandom pattern of emission, or a combination thereof. Similarly, collector patterns may comprise continuous collection, pulsatile collection, a pseudorandom pattern of collection, or a combination thereof.

In further embodiments of the invention, the one or more emitter patterns and/or the one or more collector patterns may be predetermined, preset at installation, cyclically or non-cyclically variable, selected from or alternating between preset options, or controllable in real time. In certain embodiments, the one or more emitter patterns and/or the one or more collector patterns may be directed by electric communication, magnetic communication, a wired control path, Wi-Fi®, Internet of things, Bluetooth®, near field communication, short area networks, or a combination thereof.

In certain embodiments, the distances between elements will be different. FIG. 3 illustrates a larger distance between emitters and collectors than that illustrated in FIGS. 1 and 2. In certain other embodiments of the invention, the between the one or more emitters and the one or more collectors, the distance between one or more emitters and any other emitters, and the distance between one or more collectors and any other collectors can be much larger, and the apparatus could cover a much bigger area. In certain preferred embodiments, the one or more emitters are physically disconnected from the one or more collectors by not less than 5 feet and not more than 25 feet.

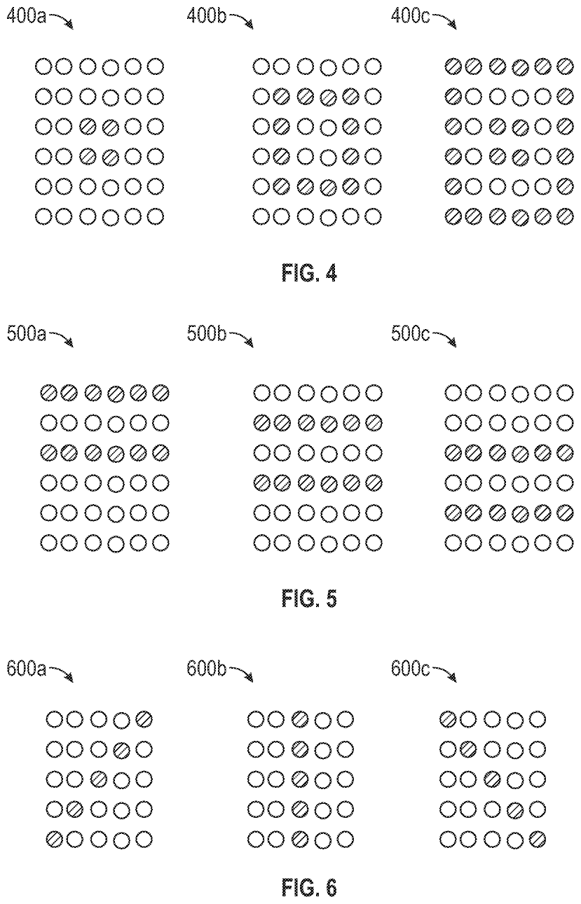

FIGS. 4-6 schematize potential temporospatial emitter patterns through illustrations of quadrilateral arrays of emitters, each emitter represented by a circle, and each emitter being in varying states of activation, which is represented by the circle having hash marks, versus deactivation, which is represented by the circle having no hash marks. All patterns disclosed herein may be pulsatile or continuous, temporary or permanent, predetermined or not, and/or cyclical or non-cyclical, including pseudo-random.

In FIG. 4, a six by six array of emitters comprises an internal small-sized quadrilateral of emitters, a middle medium-sized quadrilateral of emitters, and an outer large-sized quadrilateral of emitters. In the first state 400a, the small-sized quadrilateral of emitters is activated and the medium- and large-sized quadrilaterals of emitters are deactivated. In the second state 400b, the medium-sized quadrilateral of emitters is activated and the small- and large-sized quadrilaterals of emitters are deactivated. In the third state 400c, the small- and large-sized quadrilaterals of emitters are activated and the medium-sized quadrilateral of emitters is deactivated. This is an example pattern of quadrilaterals of emitters changing in size, and one or more than one quadrilateral of emitters can be active at the same time. The term quadrilateral as used herein can include simple quadrilateral, convex quadrilateral, concave quadrilateral, complex quadrilateral, irregular quadrilateral, trapezium, trapezoid, isosceles trapezium, isosceles trapezoid, parallelogram, rhombus, rhomb, rhomboid, rectangle, square, oblong, kite, tangential quadrilateral, tangential trapezoid, cyclic quadrilateral, right kite, harmonic quadrilateral, bicentric quadrilateral, orthodiagonal quadrilateral, equidiagonal quadrilateral, ex-tangential quadrilateral, equilic quadrilateral, quadric quadrilateral, diametric quadrilateral, dart, arrowhead, or any four-sided figure.

In FIG. 5, a six by six array of emitters comprises a first, second, third, fourth, fifth, and sixth row of emitters ordered from the top to the bottom of the page. In the first state 500a, the first and third rows of emitters are activated and the second, fourth, fifth, and sixth rows of emitters are deactivated. In the second state 500b, the second and fourth rows of emitters are activated and the first, third, fifth, and sixth rows of emitters are deactivated. In the third state 500c, the third and fifth rows of emitters are activated and the first, second, fourth, and sixth rows of emitters are deactivated. This is an example pattern of moving lines of emitters, and one or more than one line of emitters can be active at the same time.

In FIG. 6, a five by five array of emitters comprises a first, second, third, fourth, and fifth row of emitters ordered from the top to the bottom of the page and a first, second, third, fourth, and fifth column of emitters ordered from the left to the right of the page. In the first state 500a, the emitter in the first column and fifth row, the emitter in the second column and fourth row, the emitter in the third column and third row, the emitter in the fourth column and second row, and the emitter in the fifth column and first row are activated while the remaining emitters are deactivated. In the second state 600b, the emitters in the third column are activated while the remaining emitters are deactivated. In the third state 600c, the emitter in the first column and first row, the emitter in the second column and second row, the emitter in the third column and third row, the emitter in the fourth column and fourth row, and the emitter in the fifth column and fifth row are activated while the remaining emitters are deactivated. This is an example pattern of rotating lines of emitters. A next state may comprise the emitters in the third row being activated while the remaining emitters are deactivated. In certain embodiments, more than one line of emitters can be active at the same time.

Various temporal, spatial, and temporospatial patterns of activation versus deactivation of emitters and/or collectors will be readily apparent to those of ordinary skill in the art. Such patterns may involve non-shapes or shapes, whether geometric, non-geometric, polygonal, non-polygonal, one-dimensional, two-dimensional, three-dimensional, or otherwise, or a combination thereof such patterns may be cyclic or non-cyclic, and may progress in a manner that is repeating, reversing, alternating, pseudo-random, or otherwise, or a combination thereof. Such patterns may involve size that is increasing, decreasing, changing, or non-changing, shape that is changing or non-changing, or motion across space, whether translational, rotational, or otherwise, or a combination thereof. Such patterns may be pulsatile, continuous, permanent, temporary, or a combination thereof.

Example patterns include quadrilaterals or approximate quadrilaterals of one or more emitters and/or collectors optionally moving, rotating, and/or changing in size, curved shapes and/or approximately curved shapes of one or more emitters and/or collectors optionally moving, rotating, and/or changing in size, lines and/or approximate lines of one or more emitters and/or collectors optionally moving, rotating, and/or changing in size, circles or approximate circles of one or more emitters and/or collectors optionally moving, rotating, and/or changing in size, or any shape or configuration of one or more emitters and/or collectors optionally moving, rotating, and/or changing in size, or a combination thereof. Three-dimensional correlates of configurations and/or patterns discussed herein are further contemplated as additional embodiments of the present invention.

Embodiments of the present invention use several ion emission processes and some combination of electromagnetic repulsion fields, electrostatic repulsion fields, vortex air cannons, and low frequency sonic carrier waves to spread the ions evenly in layers. One embodiment spreads the produced charged particles by pulsing out the produced ion cloud in a circle, like blowing wide horizontal smoke rings—only with ions. After being injected into the air, these repetitious layers of ions uniformly spread out in a circle from electrostatic repulsion and then begin to shower down over a 2.5 meter radius in uniform distribution, creating a persistent downward dragnet effect on particulate matter within the airspace of ionic coverage. Another embodiment spreads the ion clouds in layers by physically creating and pulsing the charged particles in temporospatial patterns from geometric grids of emitters that are above an indoor space and aimed downward. Using a system of ions generated and delivered in this manner can completely clear an unoccupied indoor space of airborne particulates in only a couple of minutes or less. Once cleared of existing particulates, such system can instantly clear any introduced aerosols from breath when the indoor space becomes occupied.

In further embodiments, by timing the pulse charges of air ions and the collector plates, it is possible to engineer an equilibrium that effectively keeps a static field layered, which disrupts chaotic buildup discharge potential and overcomes potential issues caused by emergent properties in chaos complexity mathematics. Adding in ultra-dry fog in the 1-10 nanometer range provides additional physical engineering capabilities for static equilibrium and for the precipitation effect of the field by creating a multi-diameter range of charged particles that spread out in layers like a series of dragnets falling through the air and pulling captured contaminants to collectors on the floor. Simply flashing all of the emitters on and then off can create flat but separate layers of charged particles that do not heavily interact together at first, which can disrupt the buildup that leads to static discharge events by overriding the natural development of chaotic symmetry. This can prevent discharge effects in fields up to about 2-3 meters in height.

Symmetry can be set and then broken to stop the electrostatic buildup to discharge. Instead of flashing all of the emitters on and off at the same time, a simple intermittent pattern of on and off emitters can be effective over longer distances (3-4 meters). If a second or third pattern is introduced into the sequence, greater heights can be reached (>4 meters). At greater heights, random intermittent emitter outages can prevent static discharge effects even as field uniformity merges near the ground collectors. Flashing all of the emitters (or a pattern of the emitters) on and then off can work, especially if one or more additional patterns are alternated with the original pattern, enabling coverage of a greater vertical distance.

One embodiment is to alternate two (or more) different patterns next to each other to create a staggered, multipattern-shaped 3-D cloud (made from all of the patterns) that flows downward relatively intact. Another is to have a line or pattern of firing emitters that slide or rotate across the emitter grid, which creates interconnected waves of 3-D clouds flowing downward like ribbons of liquid. Embodiments can be complex or simple. In certain preferred embodiments, each subsequent pattern disrupts the symmetry of the precedent and proceeding patterns.

Additional examples not illustrated in these figures are specifically mentioned elsewhere in this disclosure. Additional examples not illustrated are contemplated by, encompassed by, and included in the present invention, and will readily come to those of ordinary skill in the art. The purpose of such patterns is to generate, emit, accelerate, collect, and/or control charged particles in patterns. Such patterns may have advantages including, but not limited to, one cloud of particles repelling another to urge the other cloud in predetermined direction, the repellent charges of multiple clouds, reducing the dispersal tendency of particles in another cloud, and/or reducing chaotic effects within and/or between clouds of particles. It will be understood that which patterns are more effective in a given installation may depend on environmental factors including, without limitation, spacing between emitters and collectors, airflow, temperature, other electric and/or magnetic fields, etc. Selection of suitable patterns can be referred to as tuning the installation. Illustrated and disclosed patterns include some commonly effective tunings in typical installations. Determining the appropriate tuning may require use of measuring devices that detect dispersal of charged particles and/or testing using control contaminants that can be placed in the environment in known concentrations and measured on collectors after a period of operation. Such tuning may be typical in several applications. Such tuning has not previously been utilized to improve efficiency of pathogen transfer prevention and mitigation using charged particles.

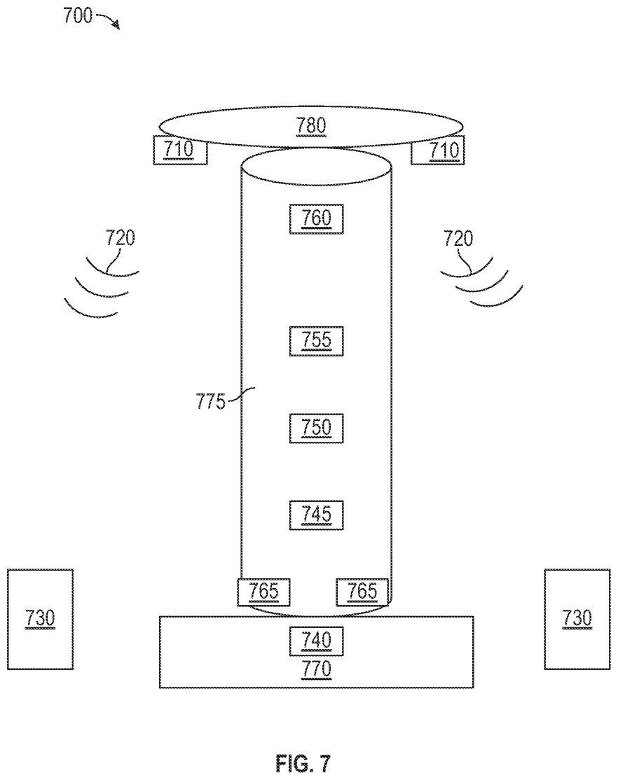

As described above, emitters, collectors, and/or other elements of the apparatuses disclosed herein may affixed to elements in the environment, such as ceilings, walls, and/or floors. Referring to FIG. 7, an alternative embodiment of a pathogen transfer prevention and mitigation apparatus a freestanding assembly is depicted. Apparatus 700 comprises base 770, shaft 775, top 780, collectors 730, and microprocessors 765. Shaft 775 is above base 770 and top 780 is above shaft 775. Base 770, shaft 775, and top 780 are connected in a vertically standing, contiguous, and portable assembly, which may be placed in a fixed location or moved as needed. Such a configuration may allow selective or positioned use in a location such as a restaurant where seating arrangements may vary and are analogous to freestanding propane heaters used in outdoor eating environments in colder weather. In apparatus 700, base 770 is approximately 22 inches in height and 18 inches in diameter, shaft 775 is approximately 52 inches in height and eight inches in diameter, and top 780 is approximately 10.089 inches in height and 36 inches in diameter.

Top 780 further comprises emitters 710, base 770 further comprises initial ionization unit 740, and shaft 775 further comprises initial transfer means 745, secondary ionization unit 750, secondary transfer means 755, and tertiary ionization unit 760.

In apparatus 700, initial ionization unit 740 converts water into an initial mixture of charged particles (not illustrated), initial transfer means 745 transfers water, the initial mixture of charged particles, or a combination thereof to secondary ionization unit 750, secondary ionization unit 750 converts water, the initial mixture of charged particles, or a combination thereof into a secondary mixture of charged particles (not illustrated), secondary transfer means 755 transfers water, the initial mixture of charged particles, the secondary mixture of charged particles, or a combination thereof to tertiary ionization unit 760, tertiary ionization unit 760 converts water, the initial mixture of charged particles, the secondary mixture of charged particles, or a combination thereof into a tertiary mixture of charged particles (not illustrated), and emitters 710 emit the charged particles 720, which may include the initial mixture of charged particles, secondary mixture of charged particles, tertiary mixture of charged particles, or a combination thereof. Collectors 730 collect charged particles, including charged particles 720. Microprocessors communicate with each other and communicate with and control initial ionization unit 740, initial transfer means 745, secondary ionization unit 750, secondary transfer means 755, tertiary ionization unit 760, emitters 710, and collectors 730.

In certain embodiments, apparatus 700 can further comprise one or more detection means (not illustrated) operable to detect ion flux, ion density, ion density flow, humidity, temperature, or a combination thereof, and communicate with microprocessors 765. In such embodiments, microprocessors 765 can control the apparatus and its elements based in whole or in part on such information detected by the one or more detection means. Such detection means may comprise a humidistat; Inkbird Humidity Controller IHC200 Humidistat; signal generator; Koolertron 60 MHz High Precision DDS Signal Generator Counter, Upgraded Dual-Channel Arbitrary Waveform Function Generator Frequency Meter 266 MSa/s (60 MHz)-ASIN: B07596133Q; Koolertron 60 MHz Embeddable Dual-Channel Function Signal Generator Counter, High Precision DDS Arbitray Waveform Generator Frequency Meter 266 MSa/s (60 MHz); HiLetgo ICL8038 Signal Generator Medium/Low Signal Frequency 10 Hz-450 KHz Triangular/Rectangular/Sine Wave Generator Module 12V to 15V; Signal Generator, KKmoon DDS Function Low Frequency Signal Generator Sine/Triangle/Square/Sawtooth/ECG/Noise Output 1 Hz˜65534 Hz; SainSmart UDB1002S DDS Signal Generator, 2 MHz Sweep Function Source Rev3.0 PC Serial Ports COMM; WHDTS Signal Generator I-Channel 1 Hz-150 KHz; PEMENOL Signal Generator, DC 3.3-30V Adjustable PWM Pulse Frequency Duty Cycle 1 Hz-150KH: Signal Generator DIY Kit, KKmoon XR2206 High Precision Function Signal Generator DIY Kit Sine/Triangle/Square Output 1 Hz-1 MH; or a combination thereof.

In certain other embodiments of the invention, the apparatus may comprise one or more initial ionization units, one or more initial transfer means, one or more secondary ionization units, one or more secondary transfer means, one or more tertiary ionization units, one or more emitters, one or more collectors, one or more microprocessors, and/or one or more detection means. In certain other embodiments of the invention, the apparatus may comprise less, more, or different components than a base, shaft, and top. In certain other embodiments of the invention, the base, shaft, and top, and/or other components, may respectively comprise different combinations, organizations, and/or arrangements of one or more initial ionization units, one or more initial transfer means, one or more secondary ionization units, one or more secondary transfer means, one or more tertiary ionization units, one or more emitters, one or more collectors, one or more microprocessors, one or more detection means, or a combination thereof. Additional embodiments will be readily apparent to those of ordinary skill in the art.

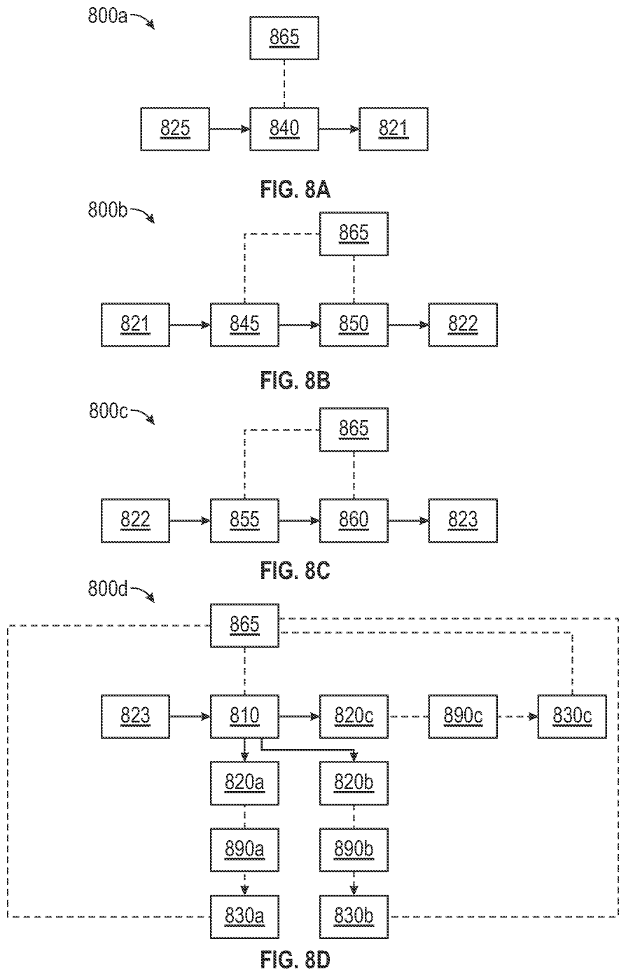

FIGS. 8A-D further explain the components and processes of pathogen transfer prevention and mitigation apparatus embodiments disclosed herein. FIG. 8A shows step 800a, where water 825 enters initial ionization unit 840 and is converted into initial mixture of charged particles 821. FIG. 8B shows the next step 800b, where initial mixture of charged particles 821 is transferred by initial transfer means 845 and converted by secondary ionization unit 850 into secondary mixture of charged particles 822. FIG. 8C shows the next step 800c, where secondary mixture of charged particles 822 is transferred by secondary transfer means 855 and converted by tertiary ionization unit into tertiary mixture of charged particles 823. Finally, FIG. 8D shows step 800d, where tertiary mixture of charged particles 823 is emitted by emitter 810 as charged particles 820a, 820b, and/or 820c. FIG. 8D shows charged particles 820a traversing a vertical distance 890a to collector 830a. FIG. 8D also shows charged particles 820b traversing vertical and horizontal distance to an obliquely positioned collector 830b. And in FIG. 8D, charged particles 830b traverse horizontal distance 890c to collector 830c.

In step 800a, initial ionization unit 840 operates via ultrasonic cavitation, physical vaporization, micro water steam impacting, or a combination thereof. In step 800b, secondary ionization unit 850 operates via plasma evaporation, pulsed laser evaporation, ionization, tuned ultraviolet light, or a combination thereof. In step 800c, tertiary ionization unit 860 comprises a carbon fiber ion generator. In certain other embodiments of the invention, the one or more initial ionization units, the one or more secondary ionization units, and/or the one or more tertiary ionization units may comprise and/or operate via other means known to those of ordinary skill in the art. Initiation of air ionization by ultrashort laser pulses can be effective. The plasma absorption is found to depend on the pulse repetition rate and is considerably stronger at 1 kHz than at 1-10 Hz.

In steps 800a and 800b, initial mixture 821 comprises superoxide ions, dry fog, or a combination thereof. In steps 800b and 800c, secondary mixture 822 comprises oxygen radicals, hydroxyl ions, superoxide ions, dry fog, ultra-dry fog, or a combination thereof. In steps 800c and 800d, tertiary mixture 823 comprises oxygen radicals, hydroxyl ions, superoxide ions, negative air ions, nanometer sized negative air ions, dry fog, ultra-dry fog, charged water particles, nanoclusters of charged water particles, or a combination thereof. In certain other embodiments of the invention, the initial, secondary, and/or tertiary mixtures of charged particles may comprise other charged particles known to those of ordinary skill in the art.

Ion Generation from water can also be effective. Water plus ultrasonic humidification can produce dry fog particles from 10-30 micrometers in diameter and the superoxide ion. This dry fog and superoxide ion combination can be sent through a set of plasma beams to evaporate and energy cleave the water droplets created by the ultrasonic step. This can produce smaller nanometer sized water molecules in addition to oxygen radicals and hydroxyl ions. Then the combination of dry fog, superoxide, oxygen radical, and hydroxyl ions can get bombarded by carbon fiber ion emitters that produce nanometer sized raw air ions, superoxide, and nano-clusters of ions and water molecules (with no measurable amounts of ozone).

In step 800b, initial transfer means 845 comprises a fan, air pump, or a combination thereof. In step 800c, secondary transfer means 855 comprises a low frequency sonic carrier wave, pulsed high velocity fan, or a combination thereof. In step 800d, emitter 810 comprises an electrostatic repulser, magnetic repulser, pulsed turbo pump fan, slow rotating frame fan, sprinkler, or a combination thereof. In certain other embodiments of the invention, the one or more initial transfer means, the one or more secondary transfer means, and/or the one or more emitters may comprise and/or operate via other means known to those of ordinary skill in the art.

In FIGS. 8A-D, microprocessor 865 acts as a controller capable of activating and deactivating initial ionization unit 840, initial transfer means 845, secondary ionization unit 850, secondary transfer means 855, tertiary ionization unit 860, emitter 810, and collectors 830a, 830b, and 830c in a coordinated pattern. Communication may be via electric communication, magnetic communication, a wired control path, Wi-Fi®, Internet of things, Bluetooth®, near field communication, short area networks, or a combination thereof. In certain other embodiments of the invention, communication may be via other means known to those of ordinary skill in the art. The result is that microprocessor 865 may be programmed to cause pulsed emissions and optionally pulsed collections in one or more predetermined patterns as previously described. Tuning maybe accomplished by varying the predetermined patterns via a user interface not illustrated, but understood in the art.

Further, elements of a pathogen transfer prevention and mitigation apparatus, such as that illustrated in FIGS. 8A-D, may comprise one or more ultrasonic transducers (such as 1.7 and 2.4 MHz ultrasonic transducers, or 113 KHz ultrasonic transducers with circuit driver boards), negative ion generators, plasma generators, signal generators, light generators, sound generators, fans, or a combination thereof.

Examples of 1.7 and 2.4 MHz ultrasonic transducers include: Ultrasonic Humidifier Vibrating Diaphragm Piezoelectric Transducer 25 mm 1.7 MHz; 10 Pcs Ultrasonic Mist Maker D20 mm Atomizing Transducer Ceramic Humidifier Lot (1.7 MHz); 2 PCS Ultrasonic Mist Maker D20 mm Atomizing Piezoelectric Transducer Ceramic (1.7 MHz); 20 mm 1.7 MHz Ultrasonic Humidifier Vibrating Diaphragm Piezoelectric Transducer; 16 mm Ultrasonic Humidifier Vibrating Diaphragm Piezoelectric Transd_KZ; Gikfun Ultrasonic Mist Maker Fogger Ceramics Discs with Wire & Sealing Ring for Arduino Aroma Diffuser DIY Kits (Pack of 5 pcs) EK1868—ASIN: B075CHT2HY (2.4 MHz); and/or Bolsen 5PCS 25 mm Ultrasonic Mist Maker Fogger Ceramics Discs with Wire & Sealing—ASIN B07QN63XF3 (1.7 MHz).

Examples of 113 KHz ultrasonic transducers with circuit driver boards include: D20 mm 113 KHz Ultrasonic Mist Maker Atomizing Fogger Ceramic Humidifier (transducer and circuit driver board); YEMIUGO 2PCS 20 mm Ultrasonic Mist Maker Transducer with PCB DC 3.7 5V 12V USB 113 KHz Mini Atomizer Fogger Water Diffuser Parts DIY Kits (transducer and circuit board driver combo) ASIN B081T25VW1; and/or 2 Pack Ultrasonic Atomization Maker 20 mm 113 KHz Mist Atomizer DIY Humidifier with PCB 3.7-12V ASIN B07V9GF44J.

Examples of negative ion generators include: AC220V Car Air Purifier Negative Ion Anion Generator Ionizer Airborne DIY Module (with 10 double heads); USA DIY 12V High Output Air Ionizer Airborne Negative Ion Anion Generator—Input Voltage: AC200-240V, AC10V, DC12V (optional); 2V High Output Air Ionizer Ionizer Airborne Negative Ion Anion Generator JG WF; Electrodepot Negative Ion Generator—High Voltage ionizer 7.5 Kv Plasma Module 110-120 VAC—ASIN B01HFHFAOG; Negative Ion Generator 12V High Density Plasma Ionizer Module; DC 12V Air Purifier Ionizer Negative Ion Anion Generator Purifier Cleaner Car single head; DC 12V Air Purifier Ionizer Negative Ion Anion Generator Purifier Cleaner_US FA single head; DC 12V Negative Ion Generator Plasma Ionizer Module Air Cleaner DIY Module New—dual head; DC 12V Negative Ion Generator Plasma Ionizer Module Air Cleaner DIY Module New—dual head; DC24v high voltage negative generator/positive ion generator/plasma ionizer generator (this one has a green ground wire) (Dongguan Nanmo Technology Co., Ltd. Manufacturer); Air Purifier Car Air Cleaner Negative Positive Ion Generator AC DC Ion Generator (this is a plasma generator and an ion generator, and it has both positive and negative so it can power both the ion emitter and the ion collector) (Guangzhou Hengxing Conduction Technology Co., Ltd. CN); AC DC Negative ion Generator 10 Million ions (Guangzhou Hengxing Conduction Technology Co., Ltd. CN); New DIY 10 Carbon Brush Head AC 220V High Output Air Ionizer Airborne Negative Ion Anion Generator (with 10 double heads) (Shenzhen Baoan Shajing Sixiang Electronic Firm); Customized DC/AC power negative ion generator (this one has a green ground wire) (Tianchang Trump Electronics Factory Manufacturer, Trading Company); and/or 2020 Negative ion wiring anion generator for ozone generator (this one has green ground wire) (Shenzhen Jeze Technology Co., Ltd.).

Examples of plasma generators include: Icstation 15 KV High Voltage Transformer Ignition Coil for DIY Electronic Pulse Arc Lighter (Pack of 2) ASIN: B073VP18C8; Icstation DC 12-30V High Voltage Arc Generator ZVS Flyback Driver Kit for SGTC Marx Generator; DEVMO 5PCS DC 3v-6v to 400 kV 400000V Boost Step-up Power Module High-Voltage Generator—ASIN B07T3XDMH8; SainSmart Zero Voltage Switching Tesla Coil Flyback Driver for Sgtc/Marx Generator/Jacob's Ladder+Ignition Coil ASIN B00ZTTVX4O; EQKIT Arc Ignition Lighter DC3-5V 3A DIY High Pressure Electronic Lighter Module 20 KHz 10 second maximum run time; ZVS Tesla Coil Boost High Voltage Generator Driver Board Induction Heating Module with High Power Bag; High Voltage Generator Electronic Lighter for USB Cigarette Lighter DC 3V-5V Arc Transformer With Coil Accessories 0.5 KV-0.8 KV (fully assemble plasma lighter assemble with battery and USB charge slot); High Voltage Generator Ultra Pulse Transformer 800-1000 KV 3.7-7.4V 4A DC Super Arc Ignition Coil Module Inverter 800 KV to 1000 KV; DIY Kits High Voltage Pressure Generator 15 KV 15000V Igniter Kit Step-Up Boost Module Coil Transformer Driver Plate Suite 2A; 15 KV High Frequency DC High Voltage Arc Ignition Generator Inverter Boost Step Up 18650 DIY Kit U Core Transformer Suite 3.7V: and/or A7—High Voltage Generator Electronic Lighter USB Cigarette Lighter DC 3V-5V Arc Trans mer With Coil Accessories (fully assemble plasma lighter with battery and USB charge slot).

Examples of signal generators include: Koolertron 60 MHz High Precision DDS Signal Generator Counter, Upgraded Dual-Channel Arbitrary Waveform Function Generator Frequency Meter 266 MSa/s (60 MHz)—ASIN: B07596133Q; Koolertron 60 MHz Embeddable Dual-Channel Function Signal Generator Counter, High Precision DDS Arbitray Waveform Generator Frequency Meter 266 MSa/s (60 MHz); HiLetgo ICL8038 Signal Generator Medium/Low Signal Frequency 10 Hz-450 KHz Triangular/Rectangular/Sine Wave Generator Module 12V to 15V; Signal Generator, KKmoon DDS Function Low Frequency Signal Generator Sine/Triangle/Square/Sawtooth/ECG/Noise Output 1 Hz˜65534 Hz; SainSmart UDB1002S DDS Signal Generator, 2 MHz Sweep Function Source Rev3.0 PC Serial Ports COMM; WIHDTS Signal Generator 1-Channel 1 Hz-150 KHz; PEMENOL Signal Generator, DC 3.3-30V Adjustable PWM Pulse Frequency Duty Cycle 1 Hz-150 KH; and/or Signal Generator DIY Kit, KKmoon XR2206 High Precision Function Signal Generator DIY Kit Sine/Triangle/Square Output 1 Hz-1 MH.

An example light generator includes: UV Germicidal Lamp LED UVC Bulb E27/E26 Home Disinfection Light Corn 130 SMD m (Bulb Life Hours: 25,000-29,999 hrs).

Example sound generators include. PYLE-PRO PPA15 Pyle 15 Inch 80 HM Woofer; and/or 12 Inch Car Subwoofer Speaker—500 Watt High Powered Car Audio Sound Component Speaker System w/1.5 Inch Kapton Bobbin Voice Coil, 8 Ohm.

An example fan includes: AC Infinity CLOUDLINE T4, Quiet 4″ Inline Duct Fan with Temperature Humidity Controller.

For example, initial ionization unit 840, secondary ionization unit 850, and/or tertiary ionization unit 860 may comprise one or more such ultrasonic transducers, negative ion generators, plasma generators, light generators, or a combination thereof. For example, microprocessors 865 may comprise one or more such signal generators. For example, initial transfer means 845 and/or secondary transfer means 855 may comprise one or more such sound generators, fans, or a combination thereof.

In certain embodiments of the invention according to the present disclosure, the one or more emitters may emit charged particles horizontally, vertically, or by any other means known to those of ordinary skill in the art. In certain embodiments of the invention according to the present disclosure, the apparatus may comprise one or more standing units, and may have one or more collectors spaced around, such as for restaurants or outside situations or settings where permanent ceiling, wall, floor, and/or other structure mounting is not feasible or less desirable. In certain embodiments of the invention according to the present disclosure, the apparatus may comprise, for example, one emitter operating in timed pulses, or a plurality of emitters operating in spatial and/or temporal patterns.

The foregoing description is of certain preferred embodiments. Those of skill in the art will readily see variations and/or improvements that may be included in alternate embodiments in light of this disclosure. The invention described herein is not intended to be limited to the preferred embodiments shown in the figures. The invention described herein is not intended to be limited to the preferred embodiments discussed in the detailed description.

1. A pathogen transfer mitigation apparatus, comprising:
one or more charged particle emitters;
an emitter power circuit to power the one or more emitters;
an emitter controller operable to selectively activate and deactivate the one or more emitters; and
one or more collectors to collect the charged particles,
wherein the emitter controller is adapted to activate and deactivate the one or more emitters in an emitter pattern that is temporal, spatial, or temporospatial.

2. The apparatus of claim 1, wherein the one or more collectors are below the one or more emitters.

3. The apparatus of claim 1, wherein the one or more emitters are selected from the group comprising oxygen radical emitter, hydroxyl ion emitter, superoxide ion emitter, dry fog emitter, ultra-dry fog emitter, emitter of negative air ions, emitter of nanometer sized negative air ions, emitter of charged water particles, emitter of nanoclusters of charged water particles, ultrasonic cavitation means, physical vaporization means, micro water stream impacting means, plasma evaporation means, pulsed laser evaporation means, ionization means, tuned ultraviolet light, carbon fiber ion generator, or a combination thereof.

4. The apparatus of claim 1, further comprising:
a collector power circuit to power the one or more collectors; and
a collector controller operable to selectively activate and deactivate the one or more collectors,
wherein the collector controller is adapted to activate and deactivate the one or more collectors in a collector pattern that is temporal, spatial, or temporospatial.

5. The apparatus of claim 4, wherein the emitter power circuit is electrically isolated from the collector power circuit.

6. The apparatus of claim 4, wherein the emitter controller and collector controller activate and deactivate in a relationship that is temporal, spatial, or temporospatial.

7. The apparatus of claim 4, wherein the emitter pattern differs from the collector pattern in time, space, or a combination thereof.

8. The apparatus of claim 4, wherein the collector pattern is selected from the group comprising continuous, pulsatile, pseudorandom, or a combination thereof, wherein the collector pattern is adapted to accelerate the rate of travel of the charged particles.

9. The apparatus of claim 1, wherein the one or more collectors comprise metallic sheets.

10. The apparatus of claim 9, wherein the metal is selected from the group comprising copper, aluminum, steel, iron, brass, bronze, zinc, nickel, graphene, copper alloy, aluminum alloy, bronze alloy, nickel alloy, iron alloy, wrought iron, cast iron, alloy steel, carbon steel, stainless steel, or a combination thereof.

11. The apparatus of claim 1, wherein the charged particles are selected from the group comprising oxygen radicals, hydroxyl ions, superoxide ions, dry fog, ultra-dry fog, or a combination thereof.

12. The apparatus of claim 1, wherein the one or more emitters are physically disconnected from the one or more collectors by not less than 5 ft and not more than 25 ft.

13. The apparatus of claim 1, wherein the one or more emitters comprise a plurality of emitters.

14. The apparatus of claim 13, wherein the emitter pattern comprises one or more quadrilaterals changing in size.

15. The apparatus of claim 14, wherein more than one quadrilateral of emitters is active at the same time.

16. The apparatus of claim 13, wherein the emitter pattern comprises one or more lines changing in size.

17. The apparatus of claim 16, wherein more than one line of emitters is active at the same time.

18. The apparatus of claim 13, wherein the emitter pattern comprises one or more lines moving.

19. The apparatus of claim 18, wherein more than one line of emitters is active at the same time.

20. The apparatus of claim 13, wherein the emitter pattern comprises one or more lines rotating.

21. The apparatus of claim 20, wherein more than one line of emitters is active at the same time.

22. The apparatus of claim 13, wherein the emitter pattern comprises one or more approximately curved shapes changing in size.

23. The apparatus of claim 22, wherein more than one approximately curved shape of emitters is active at the same time.

24. The apparatus of claim 13, wherein the emitter pattern comprises one or more circles changing in size.

25. The apparatus of claim 24, wherein more than one circle of emitters is active at the same time.

26. The apparatus of claim 1, wherein the emitter pattern is selected from the group comprising continuous, pulsatile, pseudorandom, or a combination thereof, wherein the emitter pattern is adapted to accelerate the rate of travel of the charged particles.

27. The apparatus of claim 1, wherein the one or more collectors comprise collector plates that are removable, swappable, cleanable, or a combination thereof.

28. A pathogen transfer mitigation apparatus, comprising:
an initial ionization unit;
an initial transfer means;
a secondary ionization unit;
a secondary transfer means;
a tertiary ionization unit;
one or more emitters;
one or more collectors, and
one or more microprocessors,
wherein:
the initial ionization unit converts water into an initial mixture of charged particles;
the initial transfer means transports water, the initial mixture of charged particles, or a combination thereof to the secondary ionization unit;
the secondary ionization unit converts water, the initial mixture of charged particles, or a combination thereof into a secondary mixture of charged particles;
the secondary transfer means transports water, the initial mixture of charged particles, the secondary mixture of charged particles, or a combination thereof to the tertiary ionization unit;
the tertiary ionization unit converts water, the initial mixture of charged particles, the secondary mixture of charged particles, or a combination thereof into a tertiary mixture of charged particles;
the one or more emitters emit charged particles, including the initial mixture of charged particles, secondary mixture of charged particles, tertiary mixture of charged particles, or a combination thereof;
the one or more collectors collect charged particles, including the initial mixture of charged particles, secondary mixture of charged particles, tertiary mixture of charged particles, or a combination thereof; and
the one or more microprocessors communicate with the other one or more microprocessors and communicate with and control the initial ionization unit, initial transfer means, secondary ionization unit, secondary transfer means, tertiary ionization unit, one or more emitters, and one or more collectors.

29. The apparatus of claim 28, wherein the one or more collectors are below the one or more emitters.

30. The apparatus of claim 28, wherein the initial ionization unit operates via ultrasonic cavitation, physical vaporization, micro water stream impacting, or a combination thereof.

31. The apparatus of claim 28, wherein the initial mixture of charged particles comprises superoxide ions, dry fog, or a combination thereof.

32. The apparatus of claim 28, wherein the initial transfer means comprises a fan, air pump, or a combination thereof.

33. The apparatus of claim 28, wherein the secondary ionization unit operates via plasma evaporation, pulsed laser evaporation, ionization, tuned ultraviolet light, or a combination thereof.

34. The apparatus of claim 28, wherein the secondary mixture of charged particles comprises oxygen radicals, hydroxyl ions, superoxide ions, dry fog, ultra-dry fog, or a combination thereof.

35. The apparatus of claim 28, wherein the secondary transfer means comprises a low frequency sonic carrier wave, pulsed high velocity fan, or a combination thereof.

36. The apparatus of claim 28, wherein the tertiary ionization unit comprises a carbon fiber ion generator.

37. The apparatus of claim 28, wherein the tertiary mixture of charged particles comprises oxygen radicals, hydroxyl ions, superoxide ions, negative air ions, nanometer sized negative air ions, dry fog, ultra-dry fog, charged water particles, nanoclusters of charged water particles, or a combination thereof.

38. The apparatus of claim 28, wherein the one or more emitters comprise an electrostatic repulser, magnetic repulser, pulsed turbo pump fan, slow rotating frame fan, sprinkler, or a combination thereof.

39. The apparatus of claim 28, wherein the one or more microprocessors communicate with the initial ionization unit, initial transfer means, secondary ionization unit, secondary transfer means, tertiary ionization unit, one or more emitters, one or more collectors, and the other one or more microprocessors via electric communication, magnetic communication, a wired control path, Wi-Fi®, Internet of things, Bluetooth®, near field communication, short area networks, or a combination thereof.

40. The apparatus of claim 28, further comprising one or more detection means operable to:
detect ion flux, ion density, ion density flow, humidity, temperature, or a combination thereof; and
communicate with the one or more microprocessors.

41. The apparatus of claim 40, wherein the one or more microprocessors control the initial ionization unit, initial transfer means, secondary ionization unit, secondary transfer means, tertiary ionization unit, one or more emitters, and one or more collectors on the basis of detected ion flux, ion density, ion density flow, humidity, temperature, or a combination thereof.

42. The apparatus of claim 28, wherein the initial ionization unit, initial transfer means, secondary ionization unit, secondary transfer means, tertiary ionization unit, one or more emitters, and one or more collectors are arranged vertically.

43. The apparatus of claim 28, further comprising:
a base;
a shaft above the base; and
a top above the shaft,
wherein:
the top, shaft, and base are connected in a vertically standing, contiguous, and portable assembly;
the base houses the initial ionization unit;
the shaft houses the initial transfer means, secondary ionization unit, secondary transfer means, and tertiary ionization unit; and
the top houses the one or more emitters.
